(19) United States
(12) Patent Application Publication
(10) Pub. No.: US 2026/0232919 A1
(43) Pub. Date: Aug. 13, 2026

(54) A subassembly of a medicament delivery device

(71) Applicant: SHL Medical AG, Zug (CH)

(72) Inventors: Yun Hsuan Chang, Taoyuan (TW); Meng-Jhen Chiou, Taoyuan City (TW); Wen-An Chang, Taoyuan City (TW)

(21) Appl. No.: 19/155,801

(22) PCT Filed: Feb. 5, 2024
(86) PCT No.: PCT/EP2024/052787

§ 371 Date: Aug. 12, 2025

(30) Foreign Application Priority Data
Feb. 13, 2023 (EP) 23156225.7

(51) Int. Cl. A61M 5/32; A61M 5/20

(52) U.S. Cl. CPC A61M 5/3204; A61M 2005/2073; A61M 2005/3208; A61M 2005/3254

(57) ABSTRACT

The present disclosure provides a subassembly of a medicament delivery device. The subassembly includes a housing extending axially between a proximal end and a distal end. The subassembly further includes a needle shield configured to move axially relative to the housing, the needle shield comprising a distal portion having a first guiding surface. The subassembly further includes a rotator engageable with the distal portion of the needle shield and comprising a supporting surface, the rotator having a second guiding surface configured to guide the first guiding surface to the supporting surface. The first guiding surface comprises a proximally directed portion configured to abut a distally directed portion of the supporting surface to prevent further axial movement of the needle shield in the proximal direction.

### TECHNICAL FIELD

The present disclosure generally relates to medical devices for medicament administration.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology.

For example, the medicament delivery device often comprises some type of needle shield configured to cover the needle prior to a medicament delivery operation, as well as subsequent to such medicament delivery operation. For the latter, the medicament container is typically held in a retracted position within the medicament delivery device, while the needle is prevented from being exposed in order to avoid accidental re-use of the device. Thus, the needle is configured to move relative to the needle shield. However, relative movement between components within the medicament delivery device is sometimes undesired, as such relative movement may result in unintentional actions of the medicament delivery device.

In considering these problems, the applicant has appreciated that various developments could be made to help improve the medicament delivery devices on the market today, which are set out in more detail below.

### SUMMARY

There is hence provided a subassembly of a medicament delivery device configured to expel medicament from a medicament container, the subassembly comprising: a housing extending axially between a proximal end and a distal end, a needle shield configured to move axially relative to the housing, the needle shield comprising a distal portion having a first guiding surface extending in the circumferential and/or axial direction, a rotator engageable with the distal portion of the needle shield and comprising a supporting surface configured to engage with the first guiding surface, the rotator having a second guiding surface extending in the axial direction and being configured to guide the first guiding surface to the supporting surface, wherein the first guiding surface comprises a proximally directed portion configured to abut a distally directed portion of the supporting surface to prevent further axial movement of the needle shield in the proximal direction.

The subassembly thus can provide prevention of axial movement of the needle shield in the proximal direction owing to the interaction between the supporting surface of the rotator and the first guiding surface of the needle shield. In more detail, the prevention of axial movement of the needle shield in the proximal direction is the result of the distally directed portion of the supporting surface abutting the proximally directed portion of the supporting surface and thereby preventing further axial movement of the needle shield in the proximal direction relative to the rotator. The rotator is typically axially fixed in position relative to the housing.

The interaction between the supporting surface of the rotator and the first guiding surface of the needle shield typically provides at least temporary prevention of axial movement of the needle shield in the proximal direction. For example, the at least temporary prevention of axial movement of the needle shield in the proximal direction is different to a position of the needle shield in a lock-out state of the subassembly (further described below).

The subassembly can thus provide guidance of the needle shield, e.g. as the needle shield moves axially in the proximal direction relative to the rotator, by the interaction of the first guiding surface and the second guiding surface, until the proximally directed portion of the first guiding surface abuts the distally directed portion of the supporting surface preventing further axial movement of the needle shield in the proximal direction.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment, the rotator further comprises a third guiding surface extending in the circumferential and axial direction and being configured to guide the first guiding surface from the supporting surface.

Hereby, the subassembly can thus provide guidance of the needle shield, e.g. as the needle shield moves axially in the distal direction relative to the rotator.

According to one embodiment, the distal portion comprises a radially extending guiding protrusion including the first guiding surface, and the rotator comprises a radially and axially extending guiding track including the second and third guiding surfaces. Hereby, the guiding protrusion can be guiding within the guiding track when the needle shield moves relative to the rotator. According to one embodiment, the guiding track comprises the second guiding surface and/or the third guiding surface and the supporting surface.

The guiding protrusion may extend radially outwards, e.g. from an outer surface of the distal portion, or may extend radially inwards e.g. from an inner surface of the distal portion. In addition to the radial extension, the guiding protrusion may extend in the axial and/or circumferential direction. The guiding protrusion may e.g. be formed as a knob or a rib.

The guiding track may extend radially outwards, e.g. from an outer surface of the rotator, or may extend radially inwards e.g. from an inner surface of the rotator. The guiding track may extend from a distal portion of the rotator to a proximal portion of the rotator. The guiding track may e.g. be formed as an indentation in the outer surface or inner surface of the rotator. Typically, the guiding protrusion and the supporting surface are aligned in the axial direction, at the same radial distance, when the distally directed portion of the supporting surface abuts the proximal direction portion of the supporting surface.

The rotator may be tubular, or at least comprise a tubular portion.

The distal portion of the needle shield may be tubular or may comprise at least one distally extending arm.

According to one embodiment, the rotator is arranged radially inwards of the distal portion of the needle shield. For example, the previously mentioned guiding protrusion extending from an inner surface of the distal portion is configured to be guided by the guiding track on the outer surface of the rotator.

According to one embodiment, the guiding track comprises radially and axially extending guiding walls, wherein the supporting surface forms at least a portion of the guiding walls. Herby, the distally directed portion may be comprised in said portion of the guiding walls, providing an efficient structure for abutting the proximally directed portion of the first guiding surface.

According to one embodiment, the second guiding surface is comprised in a first guiding wall. The first guiding wall may e.g. have a length extending at least in the axial direction, and a height extending in the radial direction. For example, the second guiding surface is the radially and axially extending wall surface of the first guiding wall. The first guiding wall may be referred to as a first side wall, or a first lateral side wall.

According to one embodiment, the third guiding surface is comprised of a second guiding wall. The second guiding wall may e.g. have a length extending in the axial direction and circumferential direction (e.g. extending helically), and a height extending in the radial direction. For example, the third guiding surface is the radially and helically extending wall surface of the second guiding wall. The second guiding wall may be referred to as a second side wall, or a second lateral side wall.

According to one embodiment, the second guiding surface is comprised in a first guiding wall, and the third guiding surface is comprised in a second guiding wall, as described above.

According to one embodiment, the guiding protrusion is configured to abut the portion of the guiding walls forming the supporting surface to prevent further axial movement of the needle shield in the proximal direction.

Hereby, the prevention of axial movement of the needle shield in the proximal direction is the result of the distally directed portion of the supporting surface abutting said portion of the guiding walls forming the supporting surface and thereby preventing further axial movement of the needle shield in the proximal direction relative to the rotator.

According to one embodiment, the second guiding surface is configured to guide the first guiding surface to the supporting surface upon movement of the needle shield relative to the rotator in the proximal direction into a first position.

For example, and with reference to the previously mentioned embodiment including the guiding walls, the first guiding wall with the second guiding surface is configured to guide the guiding protrusion with the first guiding surface. Thus, as the needle shield and the guiding protrusion moves axially in the proximal direction, the first guiding surface abuts, or slides along, the second guiding surface of the first guiding wall. When the protrusion and the first guiding surface reach the supporting surface, the needle shield is prevented from moving further in the proximal direction as previously described, and is thus arranged in its first position. The first position of the needle shield may thus be defined as that the first guiding surface abuts the supporting surface, or that the proximal direction portion of the first guiding surface abuts the distally directed portion of the supporting surface.

According to one embodiment, the third guiding surface is configured to guide the first guiding surface from the supporting surface upon movement of the needle shield relative to the rotator in the distal direction from the first position into a second position. Hereby, the needle shield may be moved from its first position by being moved distally.

For example, and with reference to the previously mentioned embodiment including the guiding walls, the second guiding wall with the third guiding surface is configured to guide the guiding protrusion with the first guiding surface. Thus, as the needle shield and the guiding protrusion moves axially in the distal direction, the first guiding surface abuts, or slides along, the third guiding surface of the second guiding wall. When the needle shield and the guiding protrusion is moved in the distal direction, the first guiding surface is brought away from the supporting surface, i.e. is distant from the supporting surface. The second position of the needle shield may e.g. be defined as the position in which the needle shield is prevented from moving further in the distal direction.

According to one embodiment, the sub-assembly is configured to move from: a primed state in which the needle shield is arranged in its first position and the proximally directed portion of the first guiding surface abuts the distally directed portion of the supporting surface to prevent further axial movement of the needle shield relative to the rotator in the proximal direction; into a delivery state in which the needle shield is arranged in its second position and the proximally directed portion of the first guiding surface is distant from the distally directed portion of the supporting surface to allow further axial movement of the needle shield in the proximal direction.

According to one embodiment, the needle shield is configured to move axially in the distal direction and engage the rotator to transmit the axial movement of the needle shield into a rotational movement of the rotator upon movement of the subassembly from the primed state to the delivery state. For example, during the movement of the subassembly from the primed state to the delivery state, the needle shield is moved from its first position into its second position.

According to one embodiment, the circumferentially and axially extending third guiding surface is configured to interact with the first guiding surface to transmit the axial movement of the needle shield into the rotational movement of the rotator.

For example, and with reference to the previously mentioned embodiment including the guiding walls, the second guiding wall with the third guiding surface is circumferentially and axially extending to guide the guiding protrusion with the first guiding surface. That is, as the guiding protrusion slides along the second guiding wall in the axial and circumferential direction, the rotator is rotated relative to the needle shield. The circumferentially and axially extending third guiding surface may e.g. be helically extending.

According to one embodiment, the sub-assembly is further configured to move from the delivery state into a lock-out state in which the needle shield is arranged in its furthermost proximal direction relative to the rotator. Typically, in the lock-out state, the needle shield is arranged to encompass and protect, a needle of the medicament container.

According to one embodiment, the rotator comprises a fourth guiding surface extending in the axial direction and being configured to guide the first guiding surface upon axial movement of the needle shield relative to the rotator in the proximal direction from the second position into a third position upon movement of the subassembly into the lock-out state.

According to one embodiment, the fourth guiding surface is comprised of a third guiding wall. For example, and with reference to the previously mentioned embodiment including the guiding walls, the third guiding wall with the fourth guiding surface is configured to guide the guiding protrusion with the first guiding surface. Thus, as the needle shield and the guiding protrusion moves axially in the proximal direction, towards the lock-out state of the sub-assembly, the first guiding surface abuts, or slides along, the fourth guiding surface of the third guiding wall. In the lock-out state of the sub-assembly, the needle shield is arranged in its third position.

According to one embodiment, the second, third and/or fourth guiding surface form portions of the previously mentioned guiding track. For example, the second, third and/or fourth guiding surface form portions of a coherent guiding track in an outer surface of the rotator. The second guiding surface comprised in the first guiding wall may form a first portion of the guiding track, the third guiding surface comprised in the second guiding wall may form a second portion of the guiding track, and the fourth guiding surface comprised in the third guiding wall may form a third portion of the guiding track. According to an alternative embodiment, the second, third and fourth guiding surface form separate portions of the guiding track.

According to one embodiment, the subassembly further comprises a first locking structure, wherein the needle shield comprises a second locking structure configured to lock with the first locking structure to prevent the needle shield from moving in the distal direction. This is typically achieved in the lock-out state of the sub-assembly. Thus, as the needle shield is moved into the third position, the second locking structure lock with the first locking structure to bring the sub-assembly into the lock-out state, and to thereby prevent the needle shield from moving in the distal direction.

According to one embodiment, the distal portion of the needle shield comprises the second locking structure.

According to one embodiment, the subassembly further comprises a medicament container carrier having a carrier wall comprising the first locking structure such that the distal portion of the needle shield lock to the medicament container carrier.

According to one embodiment, the first locking structure comprises a locking slot, and the second locking structure comprises a locking protrusion, wherein the locking protrusion is configured to snap-fit into the locking slot. The locking protrusion may e.g. be comprised of a proximally extending leg configured to move towards, and into, the locking slot to lock thereto. The locking protrusion may comprise at least one flexible locking tongue, configured to flex radially or circumferentially inwards during movement into the flexible locking slot and to radially or circumferentially flex outwards upon passing the locking slot.

According to one embodiment, the first locking structure comprises a flexible locking arm, and the second locking structure comprises a locking edge, wherein the flexible locking arm is configured to snap-fit with the locking edge. The flexible locking arm may e.g. be configured to move towards, and over, the locking edge to lock thereto. The flexible locking arm may comprise at least one locking hook, configured to flex radially or circumferentially outwards during movement over the locking edge and to flex radially or circumferentially inwards upon passing the locking edge.

According to one embodiment, the housing comprises the first locking structure such that the distal portion of the needle shield locks to the housing.

According to one embodiment, the first locking structure comprises a flexible blocking arm and an adjacent locking recess, and the second locking structure comprises a locking brim, wherein the locking brim is configured to be arranged in the locking recess such that flexible blocking arm locks the locking brim. The locking brim may e.g. be configured to move towards, and into, the locking recess to lock therein to the flexible blocking arm. The flexible blocking arm may comprise at least one blocking surface, and be configured to flex radially or circumferentially outwards during movement relative to the locking brim, and to flex radially or circumferentially inwards as the locking brim fits into the locking recess.

According to one embodiment, the subassembly further comprises: a pre-tensioned plunger rod operably arranged to, upon axial movement in the proximal direction, act on the medicament container for expelling a medicament, the plunger rod comprising a first holding surface having a proximally directed portion, wherein the subassembly comprises a second holding surface having a proximally directed portion configured to abut the proximally directed portion of the first holding surface to hold the plunger rod in the pre-tensioned position and prevent the plunger rod from moving axially in the distal direction.

The plunger rod may e.g. be biased by a drive spring. Thus, and according to one embodiment, the sub-assembly further comprises a drive spring biasing the plunger rod towards the proximal end of the housing.

According to one embodiment, the rotator comprises the second holding surface, wherein the proximally directed portion of the first holding surface is configured to be moved to pass the distally directed portion of the second holding surface to release the plunger rod from its pre-tensioned position and thereby allow the plunger rod to move axially in the distal direction to act on the medicament container. This is e.g. achieved during rotation of the rotator as previously described, typically during the movement of the subassembly from the primed state to the delivery state, and as the needle shield is moved from its first position into its second position. In other words, the rotator and the distally directed portion of the second holding surface are configured to abut the proximally directed portion of the first holding surface in the primed state to hold the plunger rod in the pre-tensioned position.

According to one embodiment, the rotator comprises an edge extending radially inwards from an inner surface of the rotator, the edge comprising the distally directed portion of the second holding surface.

According to one embodiment, the subassembly further comprises a holder arranged between the housing and the rotator, wherein the holder comprises the second holding surface, and wherein the proximally directed portion of the first holding surface is configured to be moved to pass the distally directed portion of the second holding surface to release the plunger rod from its pre-tensioned position and thereby allow the plunger rod to move axially in the distal direction to act on the medicament container.

According to one embodiment, the subassembly further comprises an actuator configured to lock the plunger rod in its furthermost distal position, the plunger rod comprising a first abutment surface extending in the circumferential and/or axial direction and the actuator comprising a second abutment surface extending in the circumferential and/or axial direction arranged to abut the first abutment surface to prevent the plunger rod from axial movement, wherein the actuator is operatively arranged to, upon activation, move the second abutment surface distant to the first abutment surface to release the plunger rod from its furthermost distal position. In the furthermost distal position, the plunger rod is typically biased by the previously mentioned drive spring. Thus, the previously mentioned pre-tensioned position may be referred to as an intermediate position. Thus, as the plunger rod is released from its furthermost distal position by activating the actuator, the plunger rod is moved to the pre-tensioned position where it is held by the first and second holding surfaces.

There is according to a second aspect provided a medicament delivery device for expelling medicament from a medicament container, the medicament delivery device comprising a sub-assembly according to the first aspect of the invention.

Effects and features of the second aspect are largely analogous to those described above in connection with the first aspect. Embodiments mentioned in relation to the first aspect are largely compatible with the second aspect.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a medicament delivery device according to embodiments of the present disclosure;

FIG. 2 is an exploded view of the medicament delivery device of FIG. 1 according to embodiments of the present disclosure;

FIG. 3 is a perspective view of the needle shield of FIG. 2 according to embodiments of the present disclosure;

FIG. 4 is a perspective view of the rotator of FIG. 2 according to embodiments of the present disclosure;

FIG. 5 is a perspective view of the medicament container carrier of FIG. 2 according to embodiments of the present disclosure;

FIG. 6 is a perspective view of the interaction between the needle shield of FIG. 3 and the medicament container carrier of FIG. 5 according to embodiments of the present disclosure;

FIG. 7 is a perspective view of an alternative subassembly of the medicament delivery device of FIG. 1 according to embodiments of the present disclosure;

FIGS. 8A and 8B are perspective views of an alternative subassembly of the medicament delivery device of FIG. 1 according to embodiments of the present disclosure;

FIG. 9 is a perspective view of an alternative housing and needle shield according to embodiments of the present disclosure;

FIG. 10 is a perspective view of a subassembly showing the interaction between the housing and needle shield of FIG. 9 according to embodiments of the present disclosure;

FIG. 11 is a partly cross-sectional detailed view of components of the subassembly in FIGS. 1-2 according to embodiments of the present disclosure;

FIG. 12 is a partly cross-sectional detailed view of components of the subassembly in FIGS. 1-2 according to embodiments of the present disclosure;

FIG. 13 is a partly cross-sectional view of the rotator 30 of FIG. 4 according to embodiments of the present disclosure; and

FIG. 14 is a partly cross-sectional detailed view of components of an alternative subassembly of the medicament delivery device of FIG. 1 according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to elements throughout the description.

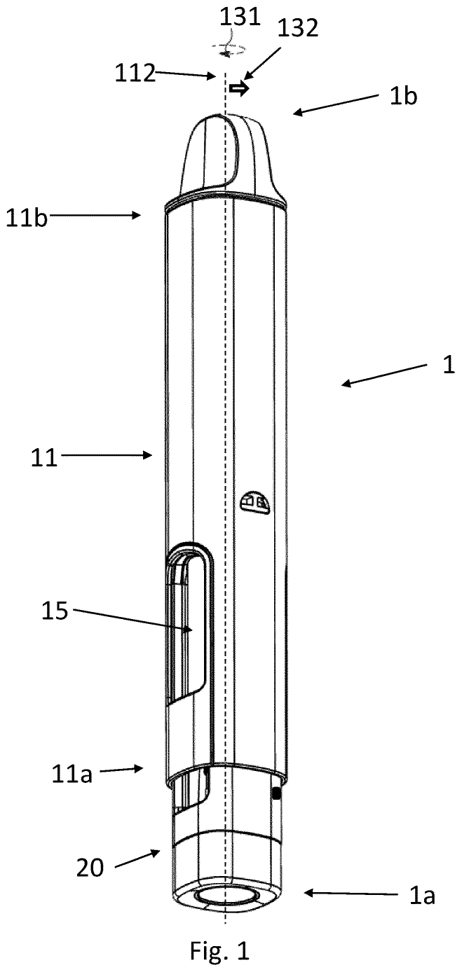

FIG. 1 shows an example of a medicament delivery device 1 such as an autoinjector according to embodiments of the present disclosure.

The medicament delivery device 1 is configured to expel medicament from a medicament container 15 via a medicament delivery member such as a needle (not shown in FIG. 1), to a patient at a dose delivery site. The medicament delivery device 1 extends from a proximal end 1a to a distal end 1b relative to the axis 112. The axis 112 is in FIG. 1 a centre axis, from which a circumferential direction 131 relative to the centre axis 112 and a radial direction 132 extending radially relative to the centre axis 112, can be defined. The medicament delivery device 1 comprises a housing 11 extending axially between a proximal end 11a and a distal end 11b, and a needle shield 20 extending at least partly proximal of the proximal end 11a of the housing 11. The needle shield 20 is configured to move axially relative to the housing 11. For example, the needle shield 20 may be moved from a protracted position as shown in FIG. 1, into a retracted position in which the needle shield 20 is received further into the housing 11, e.g. in order to expose the needle of the medicament container 15.

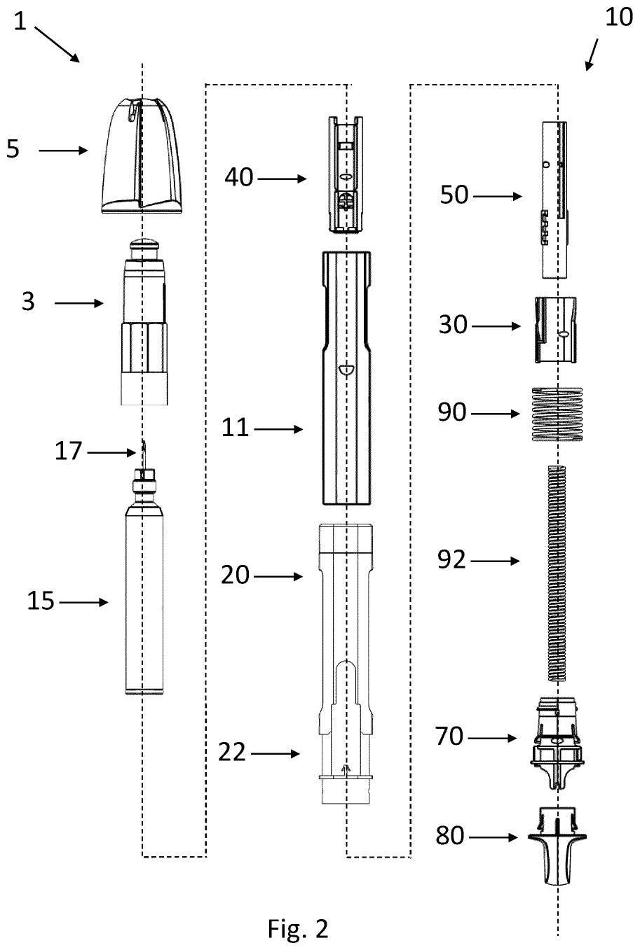

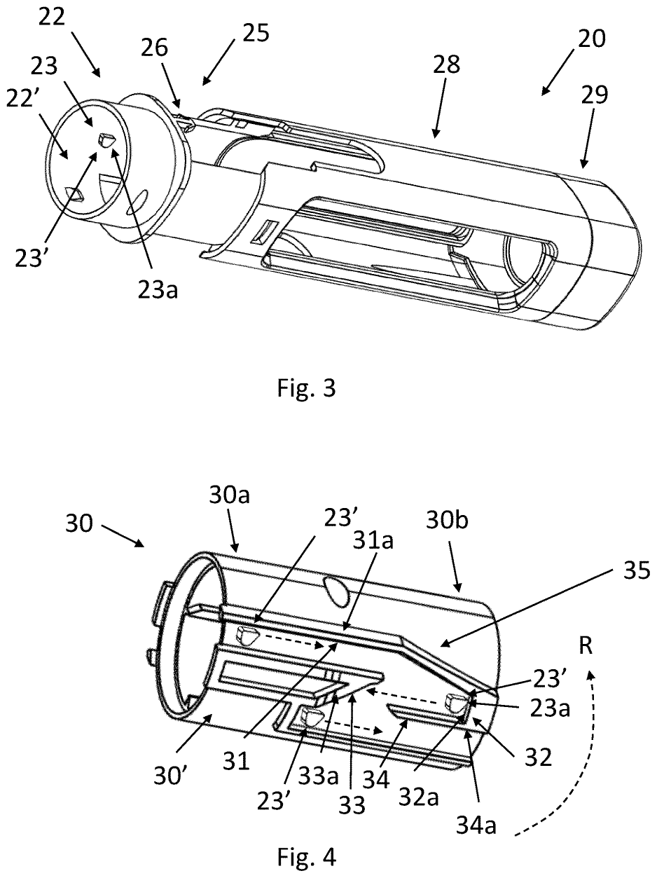

FIG. 2 is an exploded view of the medicament delivery device 1 of FIG. 1, and a subassembly 10 of the medicament delivery device 1. The subassembly 10 may comprise the housing 11 and the needle shield 20 described with reference to FIG. 1. Moreover, the subassembly 10 may comprise a rotator 30 configured to interact with the needle shield 20, better shown in FIGS. 3 and 4. The housing 11, the needle shield 20 and/or the rotator 30 may be tubular, or at least partly tubular.

The medicament delivery device 1, or the subassembly 10, may further comprise the medicament container 15 comprising a needle 17. The needle 17 extends from a proximal portion of the medicament container 15.

The medicament delivery device 1, or the subassembly 10, may further comprise a cap 5 removably attachable to the housing 11 at the proximal end 11a of the housing 11.

The medicament delivery device 1, or the subassembly 10, may further comprise a needle cap 3 arranged to protect the needle 17 of the medicament container 15. The cap 5 typically comprises an internal structure for interacting with and locking to, the needle cap 3. The internal structure may be configured to remove the needle cap 3 upon cap removal. The medicament delivery device 1 may further comprise a syringe stopper and a syringe support (not shown).

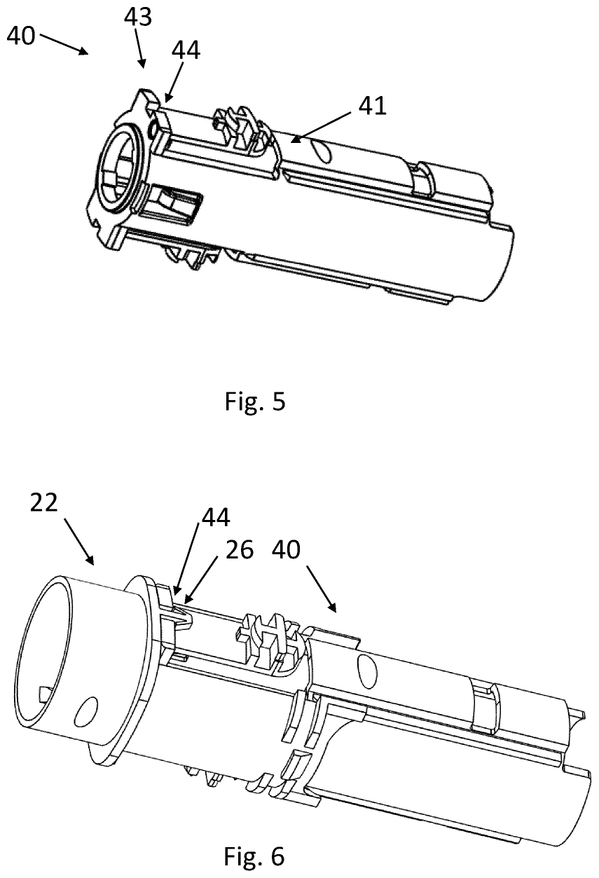

The medicament delivery device 1, or the subassembly 10, may further comprise a medicament container carrier 40. The medicament container carrier is configured to house the medicament container 15, better shown in FIG. 5. The medicament container carrier 40 may be tubular, or at least partly tubular.

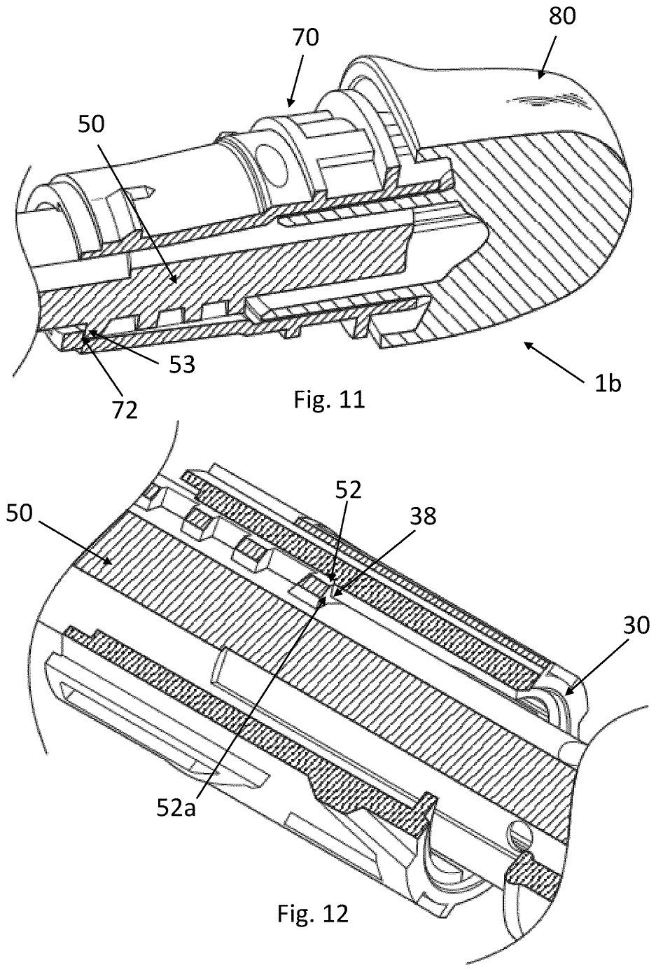

The medicament delivery device 1, or the subassembly 10, may further comprise a plunger rod 50. The plunger rod 50 is operably arranged to, upon axial movement in the proximal direction, act on the medicament container 15 for expelling a medicament. The plunger rod 15 is exemplified in e.g. FIGS. 11-12. The plunger rod 50 may e.g. be cylindrical shaped.

The medicament delivery device 1, or the subassembly 10, may further comprise an actuator 70. The actuator 70 is configured to lock the plunger rod 50 in its furthermost distal position, better shown in FIG. 11.

The medicament delivery device 1, or the subassembly 10, may further comprise an activation grip 80. The activation grip 80 is configured to interact with the actuator 70, e.g. to release the plunger rod 50 from its furthermost distal position. The activation grip 80 may e.g. be rotatable, and may be rotated by applying an external force, e.g. from a user of the medicament delivery device 1.

The medicament delivery device 1, or the subassembly 10, may further comprise a drive spring 92. The drive spring 92 may be configured to bias the plunger rod 50 towards the proximal end 11a of the housing 11.

The medicament delivery device 1, or the subassembly 10, may further comprise a needle shield spring 90. The needle shield spring 90 may be biased between the needle shield 20 and the distal end 11a of the housing 11, biasing the needle shield towards the proximal end 11a of the housing 11.

FIG. 3 is a perspective view of the needle shield 20 of FIGS. 1-2, FIG. 4 is a perspective view of the rotator 30 of FIG. 2.

The needle shield 20 comprises a distal portion 22, a proximal portion 29 and an intermediate portion 28 arranged in between the distal portion 22 and the proximal portion 29. The distal portion 22, the proximal portion 29 and the intermediate portion 28 may be made in one piece, or they may form at least two separate parts which are interconnectable with each other. Typically, the distal portion 22, the proximal portion 29 and the intermediate portion 28 are axially and rotatably movable together.

The distal portion 22 comprises a radially extending guiding protrusion 23′ including a first guiding surface 23 extending in the circumferential and axial direction of the needle shield 20. In the embodiment of FIG. 3, the distal portion 22 is tubular and comprises an inner surface 22′ from which the guiding protrusion 23′ radially extends inwards.

The distal portion 22 of the needle shield 20 may comprise a locking structure 25 in the form of a locking protrusion 26, described with reference to FIG. 6.

Turning to FIG. 4, the rotator 30 is shown in a more detailed view. The rotator 30 is engageable with the distal portion 22 of the needle shield 20, as will be described in the following. The rotator comprises guiding surfaces 31, 33, 34 and at least one supporting surface 32 configured to engage with the guiding protrusion 23′ and the first guiding surface 23 as the needle shield 20 is moved in relation to the rotator 30. The rotator 30 comprises a second guiding surface 31 extending in the axial direction of the rotator 30, the second guiding surface 31 is configured to guide the first guiding surface 23 to the supporting surface 32 upon movement of the needle shield 20 in the proximal direction into a first position.

The rotator 30 may further comprise a third guiding surface 33 extending in the circumferential and axial direction of the rotator 30, the third guiding surface 33 is configured to guide the first guiding surface 23 from the supporting surface 32 upon movement of the needle shield 20 in the distal direction from the first position to a second position.

Moreover, the rotator 30 may further comprise a fourth guiding surface 34 extending in the axial direction of the rotator 30, the fourth guiding surface 34 is configured to guide the first guiding surface 23 upon movement of the needle shield 20 in the proximal direction from the second position into a third position.

In the embodiment of FIG. 4, the rotator 30 comprises a radially and axially extending guiding track 35 including the second, third and fourth guiding surfaces 31, 33, 34 for guiding the guiding protrusion 23′ of the needle shield 20. In more detail, the guiding track 35 comprises radially and axially extending guiding walls 31a, 33a, 34a forming a guiding channel in which the guiding protrusion 23′ may be guided. Thus, the second, third and fourth guiding surfaces 31, 33, 34 are comprised of the guiding walls 31a, 33a, 34a. As shown in FIG. 4, the guiding walls 31a, 33a, 34a, are extending radially outwards from an outer surface 30′ of the rotator 30, such that a first guiding wall 31a comprises the second guiding surface 31, a second guiding wall 33a comprises the third guiding surface 33 and a third guiding wall 34a comprises the fourth guiding surface 34.

The guiding track 35 extends from a distal portion 30a of the rotator 30 to a proximal portion 30b of the rotator 30. Thus, the guiding protrusion 23′ of the needle shield 20 may be guided from the distal portion 30a to the proximal portion 30b of the rotator 30. As shown in FIG. 4, the guiding protrusion 23′ may be arranged at the distal portion 30a of the rotator 30 in a default position of the needle shield 20. As the needle shield 20 moves axially in the proximal direction from the default position, the guiding protrusion 23′ is guided by the guiding track 35 and the first guiding wall 31a and the first guiding surface 31 into the first position of the needle shield. In the first position of the needle shield 20, the guiding protrusion 23′ abuts the supporting surface 32.

In more detail, the first guiding surface 23 of the guiding protrusion 23′ comprises a proximally directed portion 23a. Moreover, the supporting surface 23 comprises a distally directed portion 32a. In the first position of the needle shield 20, the proximally directed portion 23a abuts the distally directed portion 32a of the supporting surface 32 to prevent further axial movement of the needle shield 20 in the proximal direction. That is, owing to the interaction between the supporting surface 32 of the rotator 30 and the first guiding surface 23 of the needle shield 20, prevention of axial movement of the needle shield 20 in the proximal direction is achieved. As shown in the embodiment of FIG. 4, the supporting surface 32 of the rotator 30 may form at least a portion of the guiding walls.

In the first position of the needle shield 20 in which axial movement in the proximal direction is prevented, the needle shield 20 may be moved relative to the rotator 30 in the distal direction. Thus, as the needle shield 20 moves distally from the first position to the second position, the second guiding wall 33a with the third guiding surface 33 guides the guiding protrusion 23′ with the first guiding surface 31. Thus, as the needle shield 20 and the guiding protrusion 23′ move in the distal direction, the first guiding surface 31 abuts, or slides along, the third guiding surface 33 of the second guiding wall 33a.

Hereby, the first guiding surface 31 is brought away from the supporting surface 32. Moreover, as the third guiding surface 33 and the second guiding wall 33a extend in the axial and circumferential direction of the rotator 30, the interaction with the first guiding surface 23 and the guiding protrusion 23′ results in the transmittal of the axial movement of the needle shield 20 into a rotational movement R of the rotator 30.

Typically, the medicament delivery device 1, or the subassembly 10, is configurable in a primed state in which the needle shield 20 is arranged in its first position and the proximally directed portion 23a of the first guiding surface 23 abut the distally directed portion 32a of the supporting surface 32 as previously described. Moreover, the medicament delivery device 1, or the subassembly 10, is configurable in a delivery state in which the needle shield 20 is arranged in its second position and the proximally directed portion 23a of the first guiding surface 23 is brought away from, i.e. is distant to, the distally directed portion 32a of the supporting surface 32, to thereby allow further axial movement of the needle shield 20 in the proximal direction.

Thus, as the medicament delivery device 1, or the subassembly 10, is moved from the primed state to the delivery state, the axial movement of the needle shield 20 is transmitted into the rotational movement R of the rotator 30.

FIG. 5 is a perspective view of the medicament container carrier 40 of FIG. 2, and FIG. 6 is a perspective view of a locking interaction between the needle shield 20 of FIG. 3 and the medicament container carrier 40.

The medicament container carrier 40 in FIG. 5 is tubular and is configured to house the medicament container 15.

The medicament container carrier 40 comprises a carrier wall 41 comprising a first locking structure 43 in the form of a locking slot 44. As described with reference to FIG. 3, the distal portion 22 of the needle shield 20 comprises a second locking structure 25 in the form of the locking protrusion 26. The locking slot 44 is configured to snap fit with the locking protrusion 26 such that the needle shield 20 locks to the medicament container carrier 40, as shown in FIG. 6. Hereby, the needle shield 20 is prevented from moving in the distal direction relative to the medicament container carrier 40. Typically, the medicament container carrier 40 is fixedly attached to the housing 11, and the needle shield 20 is thus also prevented from moving in the distal direction relative to the housing 11.

Referring to the previously mentioned primed state and delivery state, the medicament delivery device 1, or the subassembly 10, may thus be further configurable in a lock-out state in which the first locking structure 43 locks to the second locking structure 25 and the needle shield 20 is prevented from moving in the distal direction. In the lock-out state, the needle shield 20 is arranged in its furthermost proximal direction relative to the rotator 30 and the housing 11, encompassing and protecting the needle 17 (also referred to as the protracted position shown e.g. in FIG. 1).

Thus, as the medicament delivery device 1, or the subassembly 10, is moved from the delivery state to the lock-out state, the needle shield 20 is moved from the second position to the third position, guided by the third guiding wall 34a and the fourth guiding surface 34.

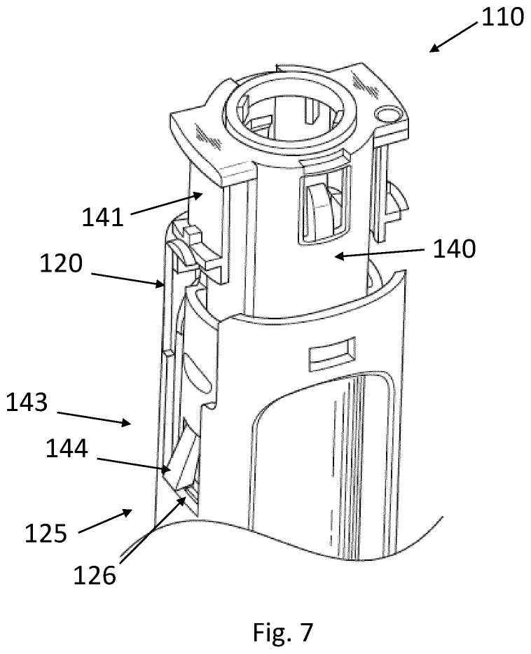

FIG. 7 is a perspective view of a locking interaction between a needle shield 120 and a medicament container carrier 140 according to an alternative example embodiment.

In FIG. 7, the medicament container carrier 140 comprises a carrier wall 141 comprising a first locking structure 143 in the form of a flexible locking arm 144. Correspondingly, the needle shield 120 comprises a second locking structure 125 in the form of a locking edge 126. The flexible locking arm 144 is configured to snap fit with the locking edge 126 such that the needle shield 120 locks to the medicament container carrier 140. Hereby, the needle shield 120 is prevented from moving in the distal direction relative to the medicament container carrier 140. As mentioned previously, the medicament container carrier 140 typically is fixedly attached to the housing 11, and the needle shield 120 is thus also prevented from moving in the distal direction relative to the housing 11.

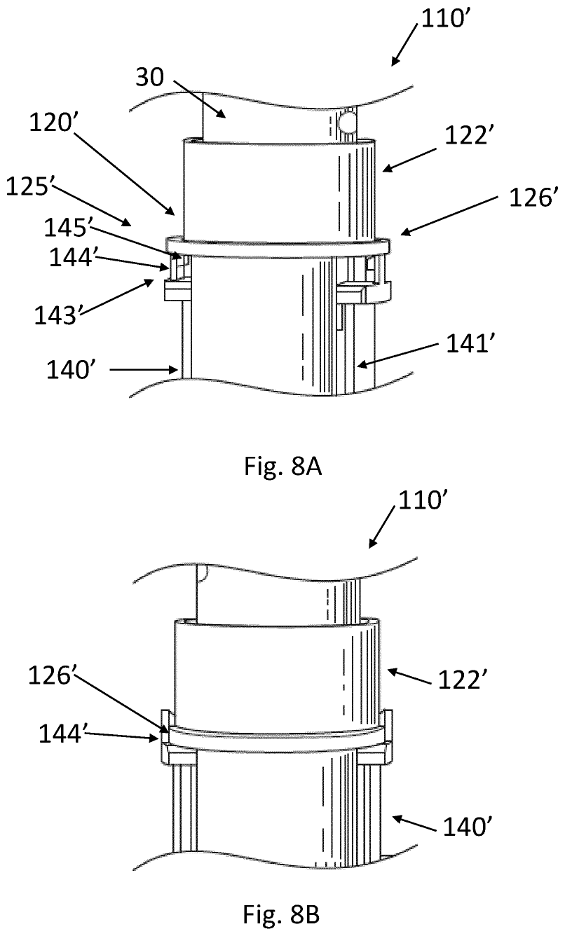

FIGS. 8A and 8B are perspective views of yet another locking interaction between a needle shield 120′ and a medicament container carrier 140′ according to an alternative example embodiment.

In FIG. 8A, the medicament container carrier 140′ comprises a carrier wall 141′ comprising a first locking structure 143′ in the form of a flexible locking arm 144′, similar to that in FIG. 7, but with the addition that the flexible locking arm 144′ comprises a locking hook 145′. In the embodiment of FIGS. 8A and 8B, the flexible locking arm 144′ extends distally from a distal portion of the medicament container carrier 140′. Correspondingly, the distal portion 122′ of needle shield 120′ comprises a second locking structure 125′ in the form of a locking edge 126′. In the embodiment of FIGS. 8A and 8B, the locking edge 126′ is an annular edge.

The flexible locking arm 144′ is configured to flex radially outwards during movement over the locking edge 126′, and to flex radially inwards upon passing the locking edge 126′, shown in FIG. 8B. Thus, the flexible locking arm 144′ is configured to snap fit with the locking edge 126′ such that the needle shield 120′ lock to the medicament container carrier 140′. Hereby, the needle shield 120′ is prevented from moving in the distal direction relative to the medicament container carrier 140′. As mentioned previously, the medicament container carrier 140′ is typically fixedly attached to the housing 11, and the needle shield 120′ is thus also prevented from moving in the distal direction relative to the housing 11.

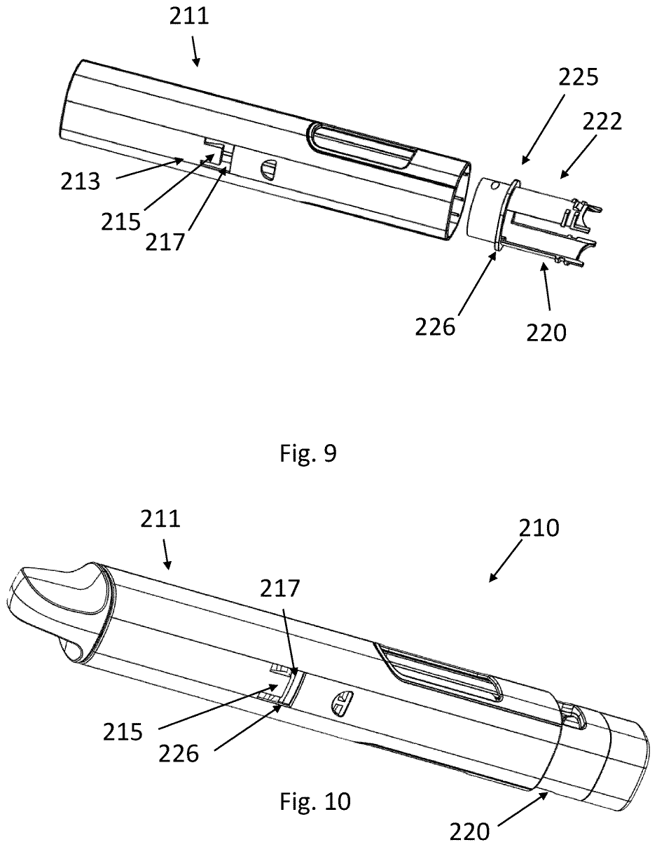

FIG. 9 is a perspective view of an embodiment of the housing 211, e.g. corresponding to the housing 11 of FIGS. 1 and 2, and a distal portion 222 of a needle shield 220. FIG. 10 is a perspective view of a subassembly 210 comprising the housing 211 and the needle shield 220 of FIG. 9.

The housing 211 comprises a first locking structure 213 in the form of a flexible blocking arm 215, or blocking tap 215, and an adjacent locking recess 217. Correspondingly, the distal portion 222 of needle shield 220 comprises a second locking structure 225 in the form of a locking brim 226. In the embodiment of FIG. 9, the locking brim 226 is an annular brim. The locking brim 226 is dimensioned to be arranged in the locking recess 217, shown in FIG. 10, such that flexible blocking arm 215 locks the locking brim 226. The locking brim 226 is configured to move over the flexible blocking arm 215 and towards, and into, the locking recess 217 to lock therein to the flexible blocking arm 215. Hereby, the needle shield 220 is prevented from moving in the distal direction relative to the housing 211.

FIG. 11 is a cross-sectional view of a distal end 1b of the medicament delivery device 1 of FIG. 2 showing the plunger rod 50, the actuator 70 and the activation grip 80. The plunger rod 50 is pre-tensioned in the housing 11, by the drive spring 90 (shown in FIG. 2), and is operably arranged to upon axial movement in the proximal direction, act on the medicament container 15 for expelling a medicament.

The actuator 70 is configured to lock the plunger rod 50 in its furthermost distal position, as shown in FIG. 11. For this reason, the plunger rod 50 may comprise a first abutment surface 53 extending in the circumferential direction, and the actuator 70 may comprise a second abutment surface 72 extending in the circumferential direction. The first abutment surface 53 typically extends radially outwards from an outer surface of the plunger rod 50, and the second abutment surface 72 typically extends radially inwards from an inner surface of the actuator 70. As the second abutment surface 72 is arranged to abut the first abutment surface 53, the plunger rod 50 is prevented from moving in the axial direction.

Upon activation of the actuator 70, e.g. by turning the activation grip 80 causing the actuator 70 to rotate, the second abutment surface 72 is moved away from the first abutment surface 53 whereby the plunger rod 50 is released from its furthermost distal position.

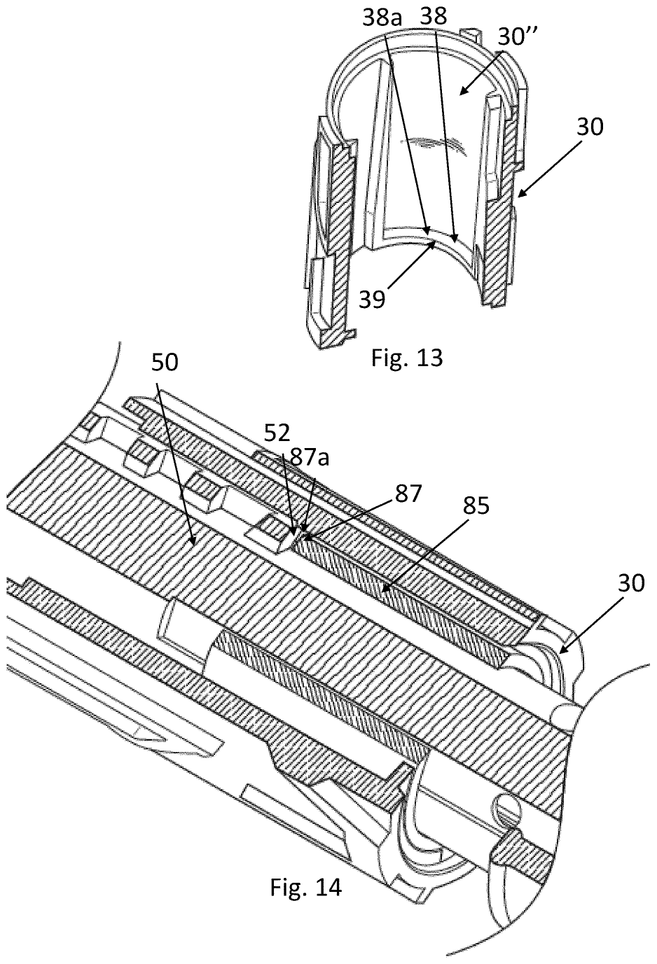

FIG. 12 is a cross-sectional view of the plunger rod 50 of FIG. 11, and the rotator 30 of FIGS. 2 and 4. The rotator 30 is configured to hold the plunger rod in a pre-tensioned position, as shown in FIG. 12. For this reason, the plunger rod 50 comprises a first holding surface 52 having a proximally directed portion 52a, and the rotator 30 comprises a second holding surface 38 having a distally directed portion 38a. The proximally directed portion 52a of the first holding surface 52 is configured to abut the distally directed portion 38a (shown in FIG. 13) of the second holding surface 38 to hold the plunger rod 50 in the pre-tensioned position. Thus, the plunger rod 50 is prevented from moving axially in the distal direction.

In more detail, as shown in FIG. 13, the rotator 30 may comprise an edge 39 extending radially inwards from an inner surface 30″ of the rotator 30. The edge 39 comprises the distally directed portion 38a of the second holding surface 38.

Thus, as the plunger rod 50 is released from its furthermost distal position, the rotator 30 hinders further axial movement of the plunger rod 50 in the proximal direction by the interaction of distally directed portion 38a of the second holding surface 38 with the proximally directed portion 52a of the first holding surface 52 of the plunger rod 50.

The proximally directed portion 52a of the first holding surface 52 is configured to be moved to pass the distally directed portion 38a of the second holding surface 38 to release the plunger rod 50 from its pre-tensioned position and thereby allow the plunger rod 50 to move axially in the distal direction to act on the medicament container 15. This is typically achieved during the rotation of the rotator 30 as previously described, during the movement of the subassembly 10 from the primed state to the delivery state.

FIG. 14 is a cross-sectional view of the plunger rod 50 of FIG. 11, and the rotator 30 of FIGS. 2 and 4, but according to an alternative example embodiment. In the embodiment of FIG. 14, a holder 85 is arranged between the housing 11 and the rotator 30. The holder 85 comprises the second holding surface 87, and a proximally directed portion 87a of the first holding surface 87 is configured to abut the proximally directed portion 52a of the first holding surface 52 of the plunger rod 50 to hold the plunger rod 50 in the pre-tensioned position. Thus, the plunger rod 50 is prevented from moving axially in the distal direction by the holder 85.

Correspondingly to the embodiment of FIG. 12, the proximally directed portion 52a of the first holding surface 52 is configured to be moved to pass the distally directed portion 87a of the second holding surface 87 of the holder 85 to release the plunger rod 50 from its pre-tensioned position and thereby allow the plunger rod 50 to move axially in the distal direction to act on the medicament container 15.

The medicament delivery devices described herein can be used for the treatment and/or prophylaxis of one or more of many different types of disorders. Exemplary disorders include, but are not limited to rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies. xemplary drugs that could be included in the injection assemblies described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), immunoglobulins (primary immune deficiencies), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present. Pharmaceutical formulations may also include separately administered or co-formulated dispersion enhancers, such as hyaluronidase.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

The inventive concept has mainly been described above with reference to a few examples. However, as is readily appreciated by a person skilled in the art, other embodiments than the ones disclosed above are equally possible within the scope of the inventive concept, as defined by the appended claims.

1-25. (canceled)

26. A subassembly of a medicament delivery device configured to expel medicament from a medicament container, the subassembly comprising:
a housing extending axially between a proximal end and a distal end;
a needle shield configured to move axially relative to the housing, the needle shield comprising a distal portion having a first guiding surface extending in the circumferential and/or axial direction; and
a rotator engageable with the distal portion of the needle shield and comprising a supporting surface configured to engage with the first guiding surface, the rotator having a second guiding surface extending in the axial direction and being configured to guide the first guiding surface to the supporting surface,
wherein the first guiding surface comprises a proximally directed portion configured to abut a distally directed portion of the supporting surface to prevent further axial movement of the needle shield in the proximal direction.

27. The subassembly of claim 26, wherein the rotator further comprising a third guiding surface extending in the circumferential and axial direction and being configured to guide the first guiding surface from the supporting surface.

28. The subassembly of claim 27, wherein the distal portion comprises a radially extending guiding protrusion including the first guiding surface, and the rotator comprises a radially and axially extending guiding track including the second and third guiding surfaces.

29. The subassembly of claim 28, wherein the guiding track comprises radially and axially extending guiding walls, and wherein the supporting surface forms at least a portion of the guiding walls.

30. The subassembly of claim 29, wherein the second guiding surface is comprised in a first guiding wall and the third guiding surface is comprised in a second guiding wall.

31. The subassembly of claim 29, wherein the guiding protrusion is configured to abut the portion of the guiding walls forming the supporting surface to prevent further axial movement of the needle shield in the proximal direction.

32. The subassembly of claim 27, wherein the second guiding surface is configured to guide the first guiding surface to the supporting surface upon movement of the needle shield relative to the rotator in the proximal direction into a first position.

33. The subassembly of claim 32, wherein the third guiding surface is configured to guide the first guiding surface from the supporting surface upon movement of the needle shield relative to the rotator in the distal direction from the first position into a second position.

34. The subassembly of claim 33, wherein the sub-assembly is configured to move from: a primed state in which the needle shield is arranged in its first position and the proximally directed portion of the first guiding surface abut the distally directed portion of the supporting surface to prevent further axial movement of the needle shield relative to the rotator in the proximal direction, into a delivery state in which the needle shield is arranged in its second position and the proximally directed portion of the first guiding surface is distant from the distally directed portion of the supporting surface to allow further axial movement of the needle shield in the proximal direction.

35. The subassembly of claim 34, wherein the needle shield is configured to move axially in the distal direction and engage the rotator to transmit the axial movement of the needle shield into a rotational movement of the rotator upon movement of the subassembly from the primed state to the delivery state.

36. The subassembly of claim 35, wherein the circumferentially and axially extending third guiding surface is configured to interact with the first guiding surface to transmit the axial movement of the needle shield into the rotational movement of the rotator.

37. The subassembly of claim 34, wherein the sub-assembly is further configured to move from the delivery state into a lock-out state in which the needle shield is arranged in its furthermost proximal direction relative to the rotator.

38. The subassembly of claim 37, wherein the rotator comprises a fourth guiding surface extending in the axial direction and being configured to guide the first guiding surface upon axial movement of the needle shield relative to the rotator in the proximal direction from the second position into a third position upon movement of the subassembly into the lock-out state.

39. The subassembly of claim 26, further comprising a first locking structure, wherein the needle shield comprises a second locking structure configured to lock with the first locking structure to prevent the needle shield from moving in the distal direction.

40. The subassembly of claim 39, wherein the distal portion of the needle shield comprises the second locking structure.

41. The subassembly of claim 39, further comprising a medicament container carrier having a carrier wall comprising the first locking structure such that the distal portion of the needle shield lock to the medicament container carrier.

42. The subassembly of claim 41, wherein the first locking structure comprises a locking slot, and the second locking structure comprises a locking protrusion, wherein the locking protrusion is configured to snap-fit into the locking slot.

43. The subassembly of claim 42, wherein the first locking structure comprises a flexible locking arm, and the second locking structure comprises a locking edge, wherein the flexible locking arm is configured to snap-fit with the locking edge.

44. The subassembly of claim 39, wherein the housing comprises the first locking structure such that the distal portion of the needle shield locks to the housing.

45. The subassembly of claim 44, wherein the first locking structure comprises a flexible blocking arm and an adjacent locking recess, and the second locking structure comprises a locking brim, wherein the locking brim is configured to be arranged in the locking recess such that flexible blocking arm locks the locking brim.

46. The subassembly of claim 26, further comprising:
a pretensioned plunger rod operably arranged to, upon axial movement in the proximal direction, act on the medicament container for expelling a medicament, the plunger rod comprising a first holding surface having a proximally directed portion, wherein the subassembly comprises a second holding surface having a proximally directed portion configured to abut the proximally directed portion of the first holding surface to hold the plunger rod in the pretensioned position and prevent the plunger rod from moving axially in the distal direction.

47. The subassembly of claim 46, wherein the rotator comprises the second holding surface, and wherein the proximally directed portion of the first holding surface is configured to be moved pass the distally directed portion of the second holding surface to release the plunger rod from its pretensioned position and thereby allow the plunger rod to move axially in the distal direction to act on the medicament container.

48. The subassembly of claim 47, wherein rotator comprises an edge extending radially inwards from an inner surface of the rotator, the edge comprising the distally directed portion of the second holding surface.

49. The subassembly of claim 46, further comprising a holder arranged between the housing and the rotator, wherein the holder comprises the second holding surface, and wherein the proximally directed portion of the first holding surface is configured to be moved pass the distally directed portion of the second holding surface to release the plunger rod from its pretensioned position and thereby allow the plunger rod to move axially in the distal direction to act on the medicament container.

50. A medicament delivery device for expelling medicament from a medicament container, the medicament delivery device comprising the sub-assembly of claim 26.
